(19) United States
(12) Patent Application Publication
(10) Pub. No.: US 2006/0064008 A1
(43) Pub. Date: Mar. 23, 2006

(54) Therapeutic use of radiation and apparatus therefor

(75) Inventor: Christopher John Moore, Manchester (GB)

Correspondence Address: WESTMAN CHAMPLIN & KELLY, P.A., SUITE 1400 - INTERNATIONAL CENTRE, 900 SECOND AVENUE SOUTH, MINNEAPOLIS, MN, 55402-3319, US

(73) Assignee: Elekta AB, Stockholm (SE)

(21) Appl. No.: 11/220,110

(22) Filed: Sep. 6, 2005

(30) Foreign Application Priority Data
Sep. 17, 2004 (GB) 0420734.6

(51) Int. Cl. A61B 5/05; A61N 5/10

(52) U.S. Cl. 600/425; 378/65

(57) ABSTRACT

A radiotherapy apparatus comprises a source of therapeutic radiation adapted to emit radiation along a therapeutic beam axis a source of diagnostic radiation adapted to emit radiation along a diagnostic beam axis, and a detector therefor, the two sources being rotateable in unison about a common axis intersecting with the therapeutic beam axis and the diagnostic beam axis; and a control unit arranged to move the therapeutic source to a first position by rotation thereof, activate the therapeutic source thereby to provide a first dose segment, de-activate the therapeutic source, rotate the sources together while the diagnostic source is active and while acquiring images from the detector, and re-activate the therapeutic source thereby to provide a second dose segment. A corresponding operation method is also disclosed, together with a reconstruction module. The invention demonstrates the feasibility of integrating cone beam imaging into normal treatment delivery, using kV projection images acquired during the gantry rotation between each treatment beam.

### CROSS-REFERENCE TO RELATED APPLICATION

The present application claims priority of Great Britain patent application Serial No. 0420734.6, filed on Sep. 17, 2004, the content of which is hereby incorporated by reference in its entirety.

### FIELD OF THE INVENTION

The present invention relates to the therapeutic use of radiation, and to apparatus therefor. It sets forth a novel method of operation of a therapeutic device and a novel apparatus and process for analysing the raw data that is obtained.

### BACKGROUND ART

Computed Tomography scanning is a well-known diagnostic technique and, in its cone beam form, involves directing a wide beam of X-rays towards and through the patient and capturing the resulting two-dimensional image on a flat panel detector behind the patient. The apparatus (source and detector) is then rotated around the patient to obtain a multiplicity of images from different directions. These images are combined via a suitable computing means in order to produce a three-dimensional representation of the internal structure of the patient.

Existing computed tomography (CT) scanners rely on a radiation source and a detector that rotate around the patient and observe the attenuation of the beam as it passes through the patient from a variety of directions. From this data, a three dimensional representation of the internal structure of the patient is computed.

CT scanners are typically fitted to therapeutic x-ray apparatus as an additional function. This allows a CT scan to be taken before and/or after the treatment process, to confirm the correct location of the patient and to record the treatment that has been given. A typical arrangement is illustrated in FIGS. 1 and 2, in which a rotateable mount 10 is shown carrying a therapeutic source 12, adapted to emit a beam 12 of megavoltage x-rays toward the rotation axis of the mount 10, along a therapeutic beam axis 16. A further source 18, this time of diagnostic radiation, emits a beam 20 kilovoltage x-rays along a diagnostic beam axis 22. The two axes 16, 22 meet on the rotation axis of the mount 10, a point referred to as the isocentre.

A flat panel detector 24 is supported by the mount 10, located opposite the diagnostic source 18 so as to intercept the diagnostic beam 20. A couch 26 is positioned just below the isocentre so as to support a patient 28 at the isocentre. Thus, the flat panel detector 24 detects the diagnostic beam 20 after attenuation by the patient 28.

The apparatus can operate in one of two modes. In a first mode, the therapeutic source 12 is active and a collimated beam of high-energy radiation is directed at the patient to destroy cancerous cells. To limit the dose applied to healthy tissue, it is common to interrupt the beam 14, rotate the mount 10 (and the source 12 with it), and direct the beam 14 towards the patient from a different direction. The beam 14 may be collimated differently to reflect the different shape of the tumour from the new direction. This may be repeated several times. In this way, the dose in the tumour is maximised and the does in healthy tissue is minimised.

In a second mode, the therapeutic beam is de-activated or blocked and the diagnostic beam 20 is activated. The mount 10 rotates steadily about the patient 28 and a number of images are captured by the flat panel detector 24. These images are passed to a suitable computing means 30 where they are reconstructed into a three-dimensional volume image.

### SUMMARY OF THE INVENTION

It is important to mimimise the amount of time taken to treat the patient. If the images are acquired before or after the treatment then this necessarily extends the time required on the machine. This invention discloses a method to acquire the images without extending the time required on the machine.

The present invention therefore provides a radiotherapy apparatus comprising a source of therapeutic radiation adapted to emit radiation along a therapeutic beam axis a source of diagnostic radiation adapted to emit radiation along a diagnostic beam axis, and a detector therefor, the two sources being rotateable in unison about a common axis intersecting with the therapeutic beam axis and the diagnostic beam axis; and a control unit arranged to move the therapeutic source to a first position by rotation thereof, activate the therapeutic source thereby to provide a first dose segment, de-activate the therapeutic source, rotate the sources together while the diagnostic source is active and while acquiring images from the detector, and re-activate the therapeutic source thereby to provide a second dose segment.

The rotation axis, the therapeutic beam axis and the diagnostic beam axis preferably intersect at a single point, to define an isocentre.

In practice, it will be preferable to provide for a plurality of dose segments and acquire images from the detector between each consecutive pair of dose segments. This will allow a greater number of images to be used.

A suitable reconstruction means will usually be required in order to produce a volume image from the acquired images. This is preferably associated with a storage means in which to place the images acquired between successive movements of the therapeutic source.

The present invention also relates to an operation method for a radiotherapy apparatus, the apparatus comprising a source of therapeutic radiation adapted to emit radiation along a therapeutic beam axis, a source of diagnostic radiation adapted to emit radiation along a diagnostic beam axis, and a detector therefor, the two sources being rotateable in unison about a common axis intersecting with the therapeutic beam axis and the diagnostic beam axis, the method comprising the steps of moving the therapeutic source to a first position by rotation thereof, activating the therapeutic source thereby to provide a first dose segment, de-activating the therapeutic source, rotating the sources together while the diagnostic source is active and while acquiring images from the detector, re-activating the therapeutic source thereby to provide a second dose segment.

Further, the invention provides a reconstruction module for a CT scanner, being adapted to accept images acquired during a first partial rotation and images acquired during a second partial rotation, and reconstruct a volume image therefrom.

The invention demonstrates the feasibility of integrating cone beam imaging into normal treatment delivery, using kV projection images acquired during the gantry rotation between each treatment beam.

### BRIEF DESCRIPTION OF THE DRAWINGS

An embodiment of the present invention will now be described by way of example, with reference to the accompanying figures in which;

FIG. 1 shows a typical therapeutic x-ray device incorporating a diagnostic cone beam CT function; and

FIG. 2 shows schematically the processing apparatus.

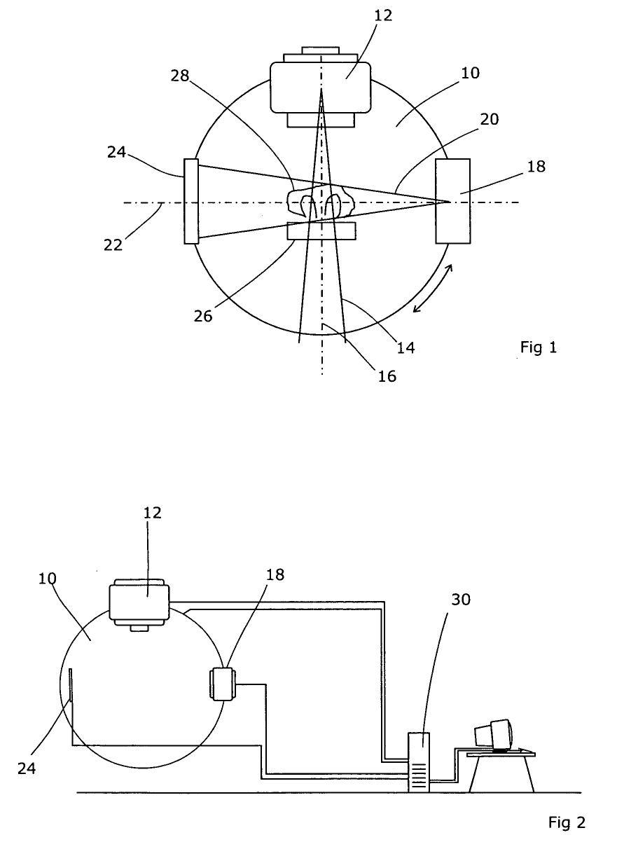

### DETAILED DESCRIPTION OF THE EMBODIMENTS

Sequences of kilo-voltage X-ray projection images of a consented bladder patient were acquired, using the Elekta Synergy™ System for two distinct acquisition techniques applied on consecutive treatment days.

In the first technique, a normal continuous gantry rotation was used after treatment for acquisition of images. These were then reconstructed to form a volume image.

According to the second technique, images were acquired between treatment beam deliveries, in a segmented gantry rotation for a four field orthogonal treatment. In such a treatment, the therapeutic source must be moved to a first position, activated to provide the first dose segment, moved to a second position, re-activated to provide the second dose segment, and so on. Each move involves a rotation of the mount 10, and according to this technique the diagnostic source is activated during this movement to acquire images for the formation of a CT dataset.

It should be noted that whilst each movement of this type generally does not involve a complete rotation through 360, in total during the complete treatment it is likely that most or all of the possible image directions will be included.

The projection sequences according to both sequences were then reconstructed using a Feldkamp cone beam back-projection algorithm to produce 256×256×256 volumes with isotropic 1 mm resolution. The two reconstructions were then compared.

Despite the different modes of acquisition, the two reconstructed volumes were remarkably similar. In particular the volume image for segmented, in-treatment acquisition was free of additional artefacts. No emphasized effects of patient movement during the extended, segmented, in-treatment acquisition were visible in the reconstruction. In-treatment acquisition according to the second technique has the potential to save about a minute per treatment fraction, thereby reducing the overall treatment time significantly to the benefit of the patient.

It is thus feasible to acquire projection images during gantry rotation between treatment beam deliveries. The quality of the reconstructed images is very similar to that of volume images acquired before or after treatment with full continuous gantry rotation. For offline verification purposes, these images are also more representative of the actual patient position during treatment. The method disclosed herein will provide verification images with decreased time and increased clinical process efficiency.

It will of course be understood that many variations may be made to the above-described embodiment without departing from the scope of the present invention. For example, the CT systems illustrated are cone beam CT systems but the invention is equally applicable to other forms of CT analysis or, indeed, to other forms of diagnostic investigation.

1. A radiotherapy apparatus comprising:
a source of therapeutic radiation adapted to emit radiation along a therapeutic beam axis;
a source of diagnostic radiation adapted to emit radiation along a diagnostic beam axis, and a detector therefor;
the two sources being rotateable in unison about a common axis intersecting with the therapeutic beam axis and the diagnostic beam axis; and
a control unit arranged to move the therapeutic source to a first position by rotation thereof, activate the therapeutic source thereby to provide a first dose segment, de-activate the therapeutic source, rotate the sources together while the diagnostic source is active and while acquiring images from the detector, and re-activate the therapeutic source thereby to provide a second dose segment.

2. Radiotherapy apparatus according to claim 1 in which the rotation axis, the therapeutic beam axis and the diagnostic beam axis intersect at a single point.

3. Radiotherapy apparatus according to claim 1 in which there are a plurality of dose segments and images are acquired from the detector between each consecutive pair of dose segments.

4. Radiotherapy apparatus according to claim 1, including a reconstruction means for producing a volume image from the acquired images.

5. Radiotherapy apparatus according to claim 4 including a storage means for the images acquired between successive movements of the therapeutic source.

6. An operation method for a radiotherapy apparatus, the apparatus comprising:
a source of therapeutic radiation adapted to emit radiation along a therapeutic beam axis;
a source of diagnostic radiation adapted to emit radiation along a diagnostic beam axis, and a detector therefor;
the two sources being rotateable in unison about a common axis intersecting with the therapeutic beam axis and the diagnostic beam axis;
the method comprising the steps of:
moving the therapeutic source to a first position by rotation thereof;
activating the therapeutic source thereby to provide a first dose segment;
de-activating the therapeutic source;
rotating the sources together while the diagnostic source is active and while acquiring images from the detector;
re-activating the therapeutic source thereby to provide a second dose segment.

7. An operation method for a radiotherapy apparatus according to claim 6, in which there are a plurality of dose segments and images are acquired from the detector between each consecutive pair of dose segments.

8. An operation method for a radiotherapy apparatus according to claim 6, in which the acquired images are passed to a reconstruction means for production of a volume image.

9. An operation method for a radiotherapy apparatus according to claim 8, in which including the acquired images are collated in a storage means between successive movements of the therapeutic source.

10. A reconstruction module for a CT scanner adapted to accept images acquired during a first partial rotation and images acquired during a second partial rotation, and reconstruct a volume image therefrom.

11. A reconstruction module for a CT scanner adapted to;
accept a first data set comprising images acquired during a first partial rotation within a plane and a second data set comprising images acquired during a second partial rotation within that plane, the second partial rotation being a continuation of the first partial rotation, and
reconstruct a volume image therefrom.
